(19) United States
(12) Patent Application Publication
(10) Pub. No.: US 2006/0025429 A1
(43) Pub. Date: Feb. 2, 2006

(54) Composition and methods for inhibiting expression of hypoxia-inducible genes

(76) Inventors: Peter B. Dervan, San Marino, CA (US); Bogdan Z. Olenyuk, Tucson, AZ (US)

Correspondence Address: FOLEY & LARDNER LLP, P.O. BOX 80278, SAN DIEGO, CA, 92138-0278, US

(21) Appl. No.: 11/146,565

(22) Filed: Jun. 7, 2005

Related U.S. Application Data
(60) Provisional application No. 60/577,901, filed on Jun. 7, 2004.

(51) Int. Cl. A61K 31/522; A61K 31/513; A61K 31/4178

(52) U.S. Cl. 514/263.2; 514/269; 514/397

(57) ABSTRACT

Methods are provided for interfering with a hypoxia-mediated transcriptional pathway using an agent that binds to a hypoxia response element and inhibits transcription of a hypoxia inducible gene associated therewith. Also provided are methods of treating a patient with a solid tumors, and compositions useful for treating a patient having solid tumors, including, for example, by administering an agent that binds to a hypoxia response element in a cell. Agents for the methods of the invention are provided including compositions comprising a pyrrole imidazole polyamide or a pharmaceutically acceptable salt or complex thereof.

### CLAIM OF PRIORITY

This application claims benefit of priority to U.S. Provisional Application 60/577,901, filed Jun. 7, 2004 which is fully incorporated by reference herein, including all figures, tables, references and charts.

### GOVERNMENT SUPPORT

This invention was made with government support under Grant No. GM57148 awarded by the National Institutes of Health. The United States government may have certain rights in this invention.

### BACKGROUND OF THE INVENTION

1. Field of the Invention

The invention relates generally to therapeutic compositions and methods and more specifically to methods of treating reducing or inhibiting translation of a hypoxia inducible gene by contacting cells with an agent that binds to an hypoxia response element associated with the gene.

### BACKGROUND INFORMATION

Cancer remains a major cause of morbidity and mortality in humans. In addition to its impact on the cancer patient and family members, cancer inflicts a great burden on society. For example, the high cost of caring for and treating cancer patients contributes to increased cost of health insurance, which, in turn, results in a higher percent of uninsured people and, consequently, an increased economic burden on government social systems when the uninsured are sick or injured. Cancer also has a significant impact on businesses due to, for example, prolonged absences of cancer patients from work.

Methods for treating cancer have improved greatly over the years. For example, improved diagnostic methods combined with better surgical techniques allow surgeons to more confidently remove a tumor, while at the same time minimizing the amount of normal tissue removed. As such, the recovery time of patients can be decreased, and psychological impact due to cosmetic trauma is reduced. However, while surgery is useful for treating patients whose tumors are localized, or have only minimally spread, for example, to local lymph nodes, it has limited usefulness for patients with metastatic disease, or with systemic cancers such as leukemia or lymphoma.

Chemotherapy is one method treatment of choice for certain types of cancers and tumors. However, chemotherapeutic treatments are generally not specific for tumor cells, instead, taking advantage of differences in proliferation rates of tumor cells as compared to corresponding normal cells. As a result, chemotherapy generally is associated with severe side effects, and can be particularly devastating to rapidly renewing tissues such as blood forming tissues and epithelial tissues.

Tumor growth is dependent on angiogenesis, the introduction of new blood vessels, for the growth and metastatic spread of solid tumors. As with almost all biologic systems, angiogenesis is a complex process that requires interaction among a variety of secreted factors, cellular receptors, activators and repressors of gene expression, and signals from local cellular environments. Because angiogenesis is important in the growth and spread of cancer, each part of the angiogenesis process is a potential target for new cancer therapies. The assumption is that if a drug can stop the tumor from receiving the supply of nutrients, the tumor will “starve” and die.

Vascular endothelial growth factor (VEGF) expression is known to be up-regulated in many tumors and is driven under hypoxia conditions. As a key regulator of angiogenesis, VEGF inhibition is an attractive therapeutic target for the treatment of diseases in which angiogenesis contributes to disease development or progression, such as for example, in tumors.

While specific agents have provided advances in cancer treatment by allowing treatment of systemic or metastatic disease without causing systemic harm to the patient, the specificity of the agents also means that they are limited to treating one or, at best, a very few different cancers. As such, unique agents are needed for every different type of cancer. Thus, there exists a need to identify methods for targeting specific a gene and/or protein that is differentially expressed or active in tumor cells, and more specifically genes or proteins which play a role in angiogenesis.

### SUMMARY OF THE INVENTION

The present invention is based upon the discovery of sequence specific DNA-binding polyamide agents which can regulate hypoxia-mediated transcriptional pathways. The DNA binding polyamide agents are designed to specifically bind to a hypoxia response element of hypoxia inducible genes. As a result of their unique binding properties, these agents reduce or inhibit expression of hypoxia inducible genes. This allows for a novel route to reducing or inhibiting angiogenesis.

In one aspect, the invention provides a composition comprising a DNA binding polyamide able to bind to a hypoxia response element and reduce expression of a hypoxia inducible gene associated therewith. The DNA binding polyamide may be a pyrrole imidazole polyamide. The polyamide may reduce or inhibit the binding of hypoxia-inducible factor-1a/aryl hydrocarbon receptor nuclear translocator (HIF-1α/ARNT). In one embodiment, the polyamide may bind DNA comprising the sequence 5′-WTWCGW-3′, wherein W is A or T.

In another aspect, the invention provides a composition comprising a pyrrole imidazole polyamide able to bind to a hypoxia response element. The pyrrole imidazole polyamide may include N-methyl pyrrole and N-methyl imidazole subunits. The pyrrole imidazole polyamide may bind DNA comprising the sequence 5′-WTWCGW-3′, wherein W is A or T.

In another aspect, the invention provides a method for interfering with a hypoxia-mediated transcriptional pathway in a cell. The method involves the step of contacting the cell with an agent which binds to a hypoxia response element. The binding of the agent and the hypoxia response element reduces or inhibits binding of a hypoxia inducible factor to the hypoxia response element, thereby reducing or inhibiting expression by a hypoxia inducible gene associated with the hypoxia response element.

In another aspect of the present invention, a method is provided for reducing the growth and/or metastatic spread of a tumor. The method involves contacting a tumor with an agent that binds to a DNA sequence encoding a hypoxia response element. By binding the hypoxia response element, the agent reduces or inhibits expression by a hypoxia inducible gene.

In yet another aspect, the invention features a method for reducing or inhibiting expression of hypoxia inducible genes by contacting a hypoxia response element of a hypoxia inducible gene with a DNA binding agent. The agent, which may be a polyamide, binds DNA of the hypoxia response element having the sequence 5′-WTWCGW-3′, wherein W is A or T. In one embodiment, the DNA binding agent may bind the DNA of an HER between nucleotide positions -900 to -1000 of the VEGF gene relative to the major site of transcription initiation (Tischer, et al., J. Biol. Chem., (1991) 266, 11947-11954, Kimura, et al., Blood, (2000) 95, 189-197).

The HER of the VEGF gene (represented by Accession Number AF095785, see Brogan, et al., Hum. Immunol., (1999) 60, 1245-1249) is located in the promoter region of the VEGF gene at nucleotide positions 1388-1393. In certain preferred embodiments, the agent binds DNA of the hypoxia response element between nucleotide positions 1330 to 1430 of the VEGF gene (represented by Accession Number AF095785), preferably between nucleotide positions 1350 to 1420, more preferably between nucleotide positions 1375 and 1400, and even more preferably between nucleotide positions 1388 and 1393.

As used herein, the term “hypoxia response element” or “HRE” refers to a short segment of nucleotides within the promoter region of a gene that is recognized by a hypoxia-inducible factor (HIF), such as HIF-1. The promoter region of a gene refers to the DNA sequences that precede a gene and contain binding sites for RNA polymerase and the transcription factors necessary for gene transcription. An HRE usually includes the short consensus core nucleotide sequence 5′-CGTG-3′, preferably an HRE includes the sequence 5′-TACGTG-3′. HIF-1 is a DNA binding heterodimer consisting HIF-1α and HIF-1β.

As used herein, the term “hypoxia-inducible factor” or “HIF”, refers to any of the transcription factors of the hypoxia-inducible factor (HIF) family. Non-limiting examples of members of hypoxia-inducible factors include HIF-1 and HIF-2.

As used herein, a “hypoxia-inducible gene” refers to a gene of which transcription is regulated by a hypoxia-inducible factor. The promoter region of a hypoxia-inducible gene may include an HRE. Generally, the expression of hypoxia-inducible genes is higher in tissues or cells under hypoxia conditions than in tissues or cells under normoxia conditions. Non-limiting examples of known hypoxia-inducible genes include vascular endothelial growth factor (VEGF), VEGF receptor-1 (VEGF-1; Flt-1), endothelin-1, endothelin-2, endothelin-3, nitric oxide synthase-2 (NOS-2), heme oxygenase-1, erythropoietin (EPO), ceruloplasmin, transferrin, transferrin receptor, insulin-like growth factor-binding protein-1, -2, and -3, insulin-like growth factor II, transforming growth factor-β3, cyclooxygenase-1 (COX-1), aldolase A and C, phosphoglycerate kinase-1 (PGK-1), phosphofructokinase (PFK), glucose transporters 1 and 3, hexokinase 1 and 2, glyceraldehyde-3-phosphate dehydrogenase (G-3-PD), enolase 1, pyruvate kinase M, lactate dehydrogenase (LDH) A, adenylate kinase 3.

As used herein, the term “hypoxia-mediated transcriptional pathway” refers to any component of the endogenous regulatory system that governs the expression of a hypoxia-inducible gene. Thus, an HRE, an HIF, a hypoxia-inducible gene, and any relevant co-transcription factors, binding partners etc., may be components of a hypoxia-mediated transcriptional pathway. The term “interfere with a hypoxia-mediated transcriptional pathway” refers to the reduction or inhibition of the function of one or more components of a hypoxia-mediated transcriptional pathway. Preferably interfering with a hypoxia-mediated transcriptional pathway results in a reduction or inhibition of the expression of a hypoxia-inducible gene.

As used herein, the term “hypoxia response element activity” refers to the ability of hypoxia inducible factor to bind to a hypoxia response element with its binding partner, aryl hydrocarbon receptor nuclear translocator (ARNT). For purposes of the present invention, it is considered that hypoxia response element activity is related, at least in part, to the level of gene expression, and the subsequent activity of the inducible gene in a hypoxia condition.

As used herein the term “agent,” when used to refer to the invention method, refers to a molecule that binds to DNA and regulates transcription of a target gene. Preferred agents of the invention are capable of permeating living cells, localizing in the nucleus and binding to the predetermined DNA sequences with affinity and sequence specificity sufficient for gene-specific disruption of a transcription factor contacting with the genomic DNA (Dervan, et al., Curr. Opin. Struct. Biol., (2003) 13, 284-299; Dudouet, et al., Chem. Biol., (2003) 10, 859-867). Specifically, an agent of the invention is capable of binding to DNA and interfering with a hypoxia-mediated transcriptional pathway. Preferred agents of the invention bind to DNA sequences corresponding to an HRE thereby reducing or inhibiting binding of an HIF to the HRE. Binding an HRE by an agent of the invention may cause a reduction or inhibition of the expression of a hypoxia-inducible gene. In certain embodiments, an agent of the invention will be less effective in reducing expression of a hypoxia-inducible gene in cells under normoxia conditions than in a cells under hypoxia conditions. Likewise, certain preferred agents of the invention may be capable of reducing the expression of a hypoxia-inducible genes only to levels similar to that observed in cells under normoxia conditions. In certain preferred embodiments, an agent of the invention will not exhibit deleterious effects on the cell growth or division of cells under normoxia conditions. Molecules that may be useful as agents in the invention include, for example, peptides (or polypeptides), polynucleotides, peptidomimetics (e.g., peptide nucleic acids, PNA) and small organic molecules (e.g., polyamides). Preferably, an agent of the invention is a small organic molecule, more preferably a polyamide. For wide applicability, agents of the invention may also include features that provide ease of synthesis, high chemical and thermal stability, and the presence of readily detectable in vivo localization markers. Synthetic polyamides containing N-methylpyrrole (Py), N-methylimidazole (Tm), and 3-hydroxypyrrole (Hp) amino acids conjugated to a fluorescent functionality represent a unique, modular molecular recognition toolkit with properties that may satisfy these criteria (Dervan, et al., Curr. Opin. Struct. Biol., (2003) 13, 284-299; Dervan, Biorg. Med. Chem., (2001) 9, 2215-2235; Gottesfeld, et al., Nature, (1997) 387, 202-205), and are examples of agents of the invention. An example of one preferred agent of the invention is polyamide 1 shown in FIG. 1.

As used herein, the term “hypoxia conditions” refers to conditions that would usually cause a cellular response typically induced in a cell by low oxygen concentrations. Likewise, the term “hypoxia-induced” refers to a cell or tissue under hypoxia conditions. Conversely, “uninduced” as used herein in reference to hypoxia conditions refers to a cell that is not under conditions which would typically cause a hypoxia response. The term “uninduced” with respect to hypoxia conditions is synonymous with the term “normoxia”. Cellular responses typically induced under hypoxia conditions are known in the art. Such responses include changes in the intracellular levels of certain components of the hypoxia-mediated transcriptional pathway. For example, whereas the intracellular levels of HIF-1β protein remain relatively stable under in cells exposed to various oxygen concentrations, the intracellular level of HIF-1α protein rapidly increases in cells exposed to low-oxygen conditions. Thus, an increase in the intracellular level of HIF-1α protein is one a marker of that can be used to determine that a cell is hypoxia-induced.

As used herein, the term “reduce or inhibit the expression of a hypoxia-inducible gene” refers to a reduction or inhibition of the transcriptional and/or translational activity of a hypoxia inducible gene caused by an agent of the invention. As such a reduction or inhibition of expression may be determined or shown by any method known in the art. Generally, such methods involve contacting an agent with a cell, contacting an agent with a tissue, or administering an agent to an animal, or the like and measuring gene transcription and or translation. Non-limiting methods for evaluating gene transcription and/or translation include measurement of mRNA levels (i.e. by PCR, competitive PCR, semi-quantitative PCR, real-time PCR, microarray-analysis, gene chip analysis, Northern Blotting and the like), promoter reporter assays (e.g., luciferase reporter assays, CAT reporter assays, β-gal reporter assays, CAT-reporter assays, GFP (e.g., green fluorescent protein) reporter assays and the like), in vivo gene expression systems (e.g., transgenic animals), and measurement of protein (e.g., Western Blotting, RIA, ELISA, and the like). A reduction of expression of a hypoxia-inducible gene may be any detectable amount of reduction of expression in a cell, tissue or animal treated with an agent as compared to an untreated control; preferably an agent causes gene expression to be reduced at least about 1.3-fold, or at least about 1.5-fold, or at least about 2-fold, or at least about 3-fold, or at least about 5-fold, or at least about 10 fold as compared to a control. It is understood that the expression of hypoxia-inducible gene is often higher in cells or tissues under hypoxia conditions (such as in tumors) than under normoxia conditions. Further, in certain embodiments, an agent of the invention may be most effective in reducing or inhibiting expression of a hypoxia-inducible gene in cells or tissues under hypoxia conditions. Similarly, in certain embodiments, preferred agents of the invention may not be capable of reducing expression of a hypoxia inducible gene to levels lower than those of corresponding cells under normoxia conditions. Thus, the reduction or inhibition of expression of a hypoxia inducible gene in a cell or tissue under hypoxia conditions may be expressed as a function relative to a corresponding cell or tissue under normoxia conditions. Accordingly “reduction or inhibition of a hypoxia-inducible gene” in a cell or tissue under hypoxia conditions may refer the reduction of gene expression to a level that approaches the level observed in a corresponding cell or tissue under normoxia conditions.

As used herein, the phrase to “hypoxia inducible gene associated with a response element” or similar such language used in connection with the invention methods refers to a response element located in the promoter region of a hypoxia inducible gene.

As used herein, the term “reduce or inhibit tumor growth” refers to a reduction or inhibition of the growth or size of a tumor. A reduction of tumor growth can be determined by a decrease in the growth rate of the tumor after contacting with an agent of the invention as compared to growth prior to contacting said tumor. A reduction of growth can be any detectable reduction in the cell growth as compared to an untreated control; however, preferably the agent causes the rate of tumor growth to be decreased by at least about 25%, or at least 50%, or at least 75%, or more preferably, growth is completely inhibited. In certain preferred embodiments, tumor size, as measured by either mass or volume, is reduced relative to the size prior to contacting with the agent of the invention. A reduction of tumor size can be any detectable reduction in the size of the tumor as compared to the tumor prior to contacting with the agent. Such methods of measuring tumor size and comparing relative sizes of the treated and untreated tumor are known in the art. Preferably, tumor size is decreased by at least about 25%, or at least 50%, or at least 75%, or more preferably, tumor size is reduced to levels below detection.

As used herein, “metastatic spread” refers to the spread of a disease process from one part of the body to another, such as for example, the appearance of tumors in parts of the body remote from the site of the primary tumor.

As used herein, the term “neoplastic cells” refer to abnormal cells that grow by cellular proliferation more rapidly than normal. As such, neoplastic cells of the invention may be cells of a benign neoplasm or may be cells of a malignant neoplasm. As used herein, the term “neoplastic disease” refers to a condition in a patient which is caused by, or associated with, the presence of neoplastic cells in the patient. Cancer is one example of a neoplastic disease. In certain aspects, the neoplastic cells are cancer cells. The cancer cells can be any type of cancer, including, for example, a carcinoma, melanoma, leukemia, sarcoma or lymphoma. Exemplary cancer cells amenable to inhibition of proliferation according to a method or composition of the invention include colon carcinoma cells, hepatocellular carcinoma cells, cervical carcinoma cells, lung epidermocarcinoma cells, mammary gland adenocarcinoma cells, pancreatic carcinoma cells, prostatic carcinoma cells, osteosarcoma cells, melanoma cells, acute promyelocytic leukemia cells, acute lymphoblastic leukemia cells, hepatocancreatico adenocarcinoma cells and Burkitt's lymphoma B cells. Neoplastic cells particularly amenable to inhibition of proliferation according to a method or composition of the invention include cells that have increased levels of expression under hypoxia conditions, especially those with increased levels of VEGFc gene expression, and cell proliferation as compared to corresponding normal cells.

As used herein, the term “tumor” refers to an abnormal growth of tissue or a neoplasm. As such, tumors usually comprise neoplastic cells, and may be benign or malignant. In certain preferred embodiments, a tumor targeted by compositions and methods of the invention may be a solid tumor. Non-limiting examples of solid tumors include lung tumor, melanoma, mesothelioma, mediastinum tumor, esophagal tumor, stomach tumor, pancreal tumor, renal tumor, liver tumor, hepatobiliary system tumor, small intestine tumor, colon tumor, rectum tumor, anal tumor, kidney tumor, ureter tumor, bladder tumor, prostate tumor, urethral tumor, testicular tumor, gynecological organ tumor, ovarian tumor, breast tumor, endocrine system tumor, carcinoma, sarcoma, germ line tumor, lymphoma, brain tumor, glioma, thyroid tumor, and central nervous system tumor.

As used herein the term “normal cell” is used broadly herein to refer to a non-neoplastic cell, including non-tumor cells. The term “corresponding normal cell” is used herein to refer to a non-neoplastic cell that is from the same type of organism as a specified neoplastic (e.g., cancer) cell. Generally, but not necessarily, a corresponding normal cell is of the same cell type as the cell from which the cancer cell was derived (e.g., normal colon epithelial cell for colon carcinoma cell).

As used herein the term “operatively linked”, “in operative linkage”, “linked” or “operatively associated” is used herein to refer to two or more molecules that, when joined together, act in concert. For example, when used in reference to a transcriptional regulatory element (e.g., a promoter) and a second nucleotide sequence (e.g., a polynucleotide encoding an siRNA), the term “operatively linked” means that the regulatory element is positioned with respect to the second nucleotide sequence such that the regulatory element functions to effect transcription of the second nucleotide sequence (e.g., a promoter effects transcription of an operatively linked coding sequence).

As used herein, the term “chemotherapeutic molecule” as used herein, refers to a chemical, or a chemical moiety, that alters the morphology or growth characteristics of neoplastic cells in culture or in vivo. Preferable chemotherapeutic molecules reduce the aberrant proliferation of neoplastic cells. Examples of chemotherapeutic molecules can include, but are not limited to DNA alkylators, topoisomerase inhibitors or histone deacetylase inhibitors. It is understood that a chemotherapeutic molecule of the invention may be conjugated or linked to another functional moiety such as a nucleic acid binding domain. Chemotherapeutic molecules can be conjugated or linked to a separate moiety, such as a DNA or RNA binding moiety, to increase the specificity of the chemotherapeutic molecules and/or decrease the toxicity or side-effects of the agent. Chemotherapeutic molecules can be DNA alkylators (e.g. chlorambucil). As used herein, the term “alkylator” means a compound that reacts with and adds an alkyl group to another molecule. In preferred embodiments, the alkylator is reactive with DNA at about 37 degrees Celsius, the alkylator is substantially inert in aqueous media, and/or the alkylator is present in a buffer and the alkylator is non-reactive with the buffer. Non-limiting examples of alkylators that may be used in the invention include cyclophosphamide, nitrosoureas, mitozolomide, anthramycin, bromoacetyl, a nitrogen mustard, clorambucil, a derivative of chlorambucil (such as a Bis(dichloroethylamino)benzene derivative), seco-CBI, mitomycin, initomycin C, or (+)-CC-1065. Seco-CBI is a precursor to 1,2,9,9a-tetrahydrocyclopropa[1,2-c]benz[1,2-e]indol-4-one (CBI), (Boger, D. L. et al. Bioorgan. Med. Chem. (1995), 3, 1429-1453; and Boger, D. L. and Johnson, D. S. Angew. Chem., Int. Ed. Engl., (1996), 35, 1438-1474) an analogue of the natural product (+)-CC-1065. CBI shows increased reactivity to DNA as well as increased stability to solvolysis (Boger, D. L. and Munk, S. A. J. Am. Chem. Soc. (1992), 114, 5487-5496). The seco agents readily close to the cyclopropane forms and have equivalent reactivity as compared to CBI, but have been shown to have longer shelf lives (Boger, D. L. et al. Bioorg. Med. Chem. Lett. (1991), 1, 55-58).

As used herein the term “about” refers to the indicated value ±10%.

### BRIEF DESCRIPTION OF THE DRAWINGS

FIG. 1 shows the chemical structure of polyamide-FITC conjugates of polyamide 1 and polyamide 2.

FIG. 2 shows a schematic representation of the VEGF promoter with the HRE site (upper) and schematic representation of match polyamide 1 targeting the HRE and mismatch polyamide 2 (bottom). Imidazole rings are represented as solid circles, pyrrole rings are represented as open circles, 3-chlorothiophene is depicted as a square and aliphatic linkers as curved lines. Half-diamonds with plus signs represent 3,3′-diamino-N-methyldipropylamine. FITC represents conjugated fluorescein isothiocyanate (isomer 1).

FIG. 3 shows a schematic representation of the number of viable cells after incubation with polyamides 1 and 2 as a function of time.

FIG. 4 shows inhibition of the expression of a VEGF-Luc reporter gene under physiological hypoxia.

FIG. 5 shows the inhibition of the expression of wild-type VEGF-Luc and mutated VEGF-M1Luc reporter genes under hypoxia conditions.

FIG. 6 shows the blocking of VEGF induction by hypoxia. (A) shows relative mRNA levels of expression of the VEGF gene as measure by real-time quantitative RT-PCR. (B) shows levels of secreted VEGF protein as measure by ELISA.

FIG. 7 shows the differential expression levels of VEGF from real-time PCR experiments.

FIG. 8 shows Venn diagrams representing the distribution of affected genes from the microarray experiments. The numbers outside the intersections represent genes uniquely affected by the individual polyamides.

### DETAILED DESCRIPTION OF THE INVENTION

The present invention relates to sequence-specific DNA-binding molecules directed against hypoxia-mediated transcriptional pathways and methods involving the use of such DNA-binding molecules. In cells and tissues, hypoxia triggers a multifaceted adaptive response that is primarily driven by the heterodimeric hypoxia-inducible factor 1 (HIF-1) (O'Rourke, et al. Eur. J. Biochem. (1996), 241:403-410). HIF-1 is a DNA-binding heterodimer consisting of HIF-1α and HIF-1β subunits. Under normal dioxygen levels, the α-subunit of HIF-1 is successively hydroxylated at proline residue 564 (Ivan, et al. Science (2001), 292:464-468), ubiquitatinated, and then degraded by the ubiquitin-proteosome system. This process, mediated by the von Hippel-Lindau tumor suppressor protein (Kaelin, W. G. Nat. Rev. Cancer, (2002), 2:673-682), is responsible for controlling levels of HIF-1α and, as a result, the transcriptional response to hypoxia (Maxwell, et al. Nature, (1999), 399:271-275). Under hypoxia conditions, HIF-1α avoids hydroxylation and accumulates. Heterodimerization with its constitutively expressed binding partner, aryl hydrocarbon receptor nuclear translocator (ARNT) (Wood, et al. J. Biol. Chem., (1996) 271:15117-15123) and binding to a cognate hypoxia response element (HRE) (O'Rourke, et al. Oncol. Res., (1997), 9:327-332) recruits the p300/CBP and SRC-1 family coactivators, which drive the expression of hypoxia-inducible genes. Examples of the hypoxia-inducible genes which may be expressed by such a mechanism are Vascular Endothelial Growth Factor and the platelet-derived growth factor B chain, as well as proteins involved in glucose metabolism, such as for example, glucose transporter GLUTI (Forsythe, et al. Mol. Cell. Biol., (1996) 16:4604-4613; Okino, et al. J. Biol. Chem., (1998) 273:23837-23843.

VEGF and its component receptors have received particular attention because of the potent ability of VEGF to stimulate endothelial cell proliferation and migration in vitro and angiogenesis in vivo. (Soker, et al., J. Biol. Chem., (1997), 272, 31582-32588; Hanahan, et al., Cell, (1996), 86, 353-364; Plate, et al., Nature, (1992), 359, 845-848; Leung, et al., Science, (1989), 246, 1306-1309). Angiogenesis, the induction of new blood vessels, is critical for the growth and metastatic spread of solid tumors. Without the creation of a dense network of highly permeable blood vessels, solid tumors fail to progress. In this respect, angiogenesis (i.e., the induction of new blood vessels) is critical for growth and metastatic spread of solid tumors. VEGF levels are up-regulated across a broad range of tumors and are involved in key aspects of cancer biology. A hallmark of many cancers, chronic hypoxia, in conjunction with activation of certain oncogenic signaling pathways, is responsible for the elevated levels of VEGF and is associated with invasion and altered energy metabolism (Kaelin, Genes Dev., (2002), 16, 1441-1445).

Elevated VEGF levels are associated with the progression of a variety of tumors and correlated to the outcome of cancer treatment and inhibition of VEGF, a downstream target of HIF, is sufficient to inhibit tumor growth in model systems. (Mrksich, et al., J. Am. Chem. Soc., (1993), 115, 9892-9899; Garparini, et al., J. Natl. Cancer Inst., (1997), 89, 139-147; Kang, et al., Oncol. Rep., (1997), 4, 381-384). To date, numerous attempts to block the activity of VEGF have been made, including the use of antibodies (Kim, et al. Nature (1993), 362, 841-844), soluble VEGF receptors (Lin, et al. Cell Growth Differ. (1998), 9,49-58), VEGF receptor antagonists (Hennequin, et al. J. Med. Chem. (1999), 42, 5369-5389), or degradation of the VEGF message through the use of antisense oligonucleotides (Shi, et al. Br. J. Cancer (2002), 87, 119-126), or by RNA interference (Zhang, et al. Biochem. Biophys. Res. Commun. (2003), 303, 1169-1178; Reich, et al. Mol. Vis. (2003), 9, 210-216). The major focus of the previous studies was inhibition of either a single target or a very limited number of targets.

Accordingly, in certain embodiments, the invention relates to agents that reduce or inhibit VEGF expression. However, unlike previous studies that focused on VEGF as the only target of inhibitory regimens, an object of the present invention are agents which interfere with a hypoxia-mediated transcriptional pathway. This pathway-specific approach is advantageous because it results in the down regulation of multiple genes by targeting a common transcription factor binding site. Preferably, interference with a hypoxia-mediated transcriptional pathway results in the reduction or inhibition of one or more hypoxia-inducible genes, such as for example, VEGF.

Thus, the present invention relates to agents which bind to an HRE of a hypoxia-inducible gene, thereby reducing or inhibiting the binding of an HIF to the HRE. Further, the invention relates to methods involving the use of such agents. In preferred embodiments binding of an agent of the invention to an HRE in a cell or tissue under hypoxia conditions results in the reduction or inhibition of the expression of a hypoxia-inducible gene associated with that HRE in the cell. Generally, the binding of the agent to the HRE is believed to reduce or inhibit the expression of the associated hypoxia inducible gene by reducing or inhibiting binding of a hypoxia-inducible factor to the HRE. For example, by binding to a HRE, an agent of the invention may function as a disrupter of binding of an HIF-1α/ARNT heterodimer to a congnate DNA sequence (i.e. an HRE) of one or more hypoxia-inducible genes. Preferably, the agent reduces the expression of at least 2, at least 4, at least 6, at least 8, or at least 10 or more hypoxia-inducible genes. In certain preferred embodiments, an agent of the invention reduces or inhibits the expression of one or more hypoxia-induced gene selected from the group consisting of vascular endothelial growth factor (VEGF), VEGF receptor-1 (VEGF-1; Flt-1), endothelin-1, endothelin-2, endothelin-3, nitric oxide synthase-2 (NOS-2), heme oxygenase-1, erythropoietin (EPO), ceruloplasmin, transferrin, transferrin receptor, insulin-like growth factor-binding protein-1, -2, and -3, insulin-like growth factor II, transforming growth factor-β3, cyclooxygenase-1 (COX-1), aldolase A and C, phosphoglycerate kinase-1 (PGK-1), phosphofructokinase (PFK), glucose transporters 1 and 3, hexokinase 1 and 2, glyceraldehyde-3-phosphate dehydrogenase (G-3-PD), enolase 1, pyruvate kinase M, lactate dehydrogenase (LDH) A, adenylate kinase 3. More preferably an agent of the invention reduces the expression of one or more hypoxia-inducible gene selected from the group consisting of vascular endothelial growth factor (VEGF), VEGF receptor-1 (VEGF-1; Flt-1), endothelin-1, endothelin-2, endothelin-3, erythropoietin (EPO), transforming growth factor-β3, aldolase C, and hexokinase 2. More preferably, an agent of the invention reduces or inhibits the expression of each of the genes vascular endothelial growth factor (VEGF), VEGF receptor-1 (VEGF-1; Flt-1), endothelin-1, endothelin-2, endothelin-3, erythropoietin (EPO), transforming growth factor-β3, aldolase C, and hexokinase 2. In certain preferred embodiments an agent of the invention reduces the expression of VEGF in a cell under hypoxia conditions by at least about 1.1-fold, more preferably at least about 1.2-fold, more preferably at least about 1.3-fold, more preferably at least about 1.4-fold, more preferably at least about 1.5-fold, more preferably at least about 1.6 fold, more preferably at least about 2-fold.

In certain preferred embodiments, an agent of the invention will have no effect, or lesser effects, on the expression of a hypoxia-inducible gene in cells or tissues (such as a tumor tissue) under normoxia conditions than in corresponding cells under hypoxia conditions. As such, the agent will specifically target such hypoxia induced cells or tissues (i.e. cells and tissues of a tumor) and will have little or no effect on gene expression in non-hypoxia cells or tissues. This property of an agent of the invention has advantages in that there will be reduced side-effects of such an agent when administered to an animal where the tumor tissues and tumor cells are under hypoxia conditions where the normal tissues and normal cells are under normoxia conditions. Accordingly, a preferred agent of the invention may reduce the levels of expression of a hypoxia-inducible gene in a cell or tissue under hypoxia conditions only to levels near, or comparable to, that of a corresponding cell or tissue under normoxia conditions. Thus, it is understood that, certain preferred agents of the invention may be not be capable of causing a reduction of the expression of any hypoxia-inducible genes in cells or tissues under normoxia conditions, even upon binding of the agent to an HRE in the cells.

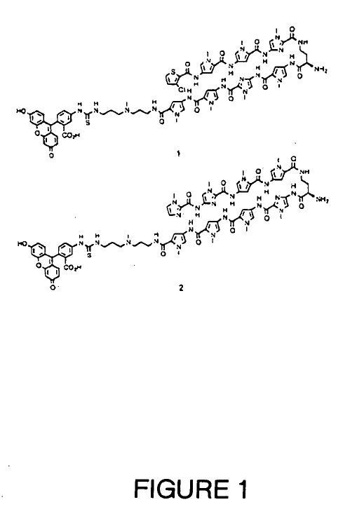

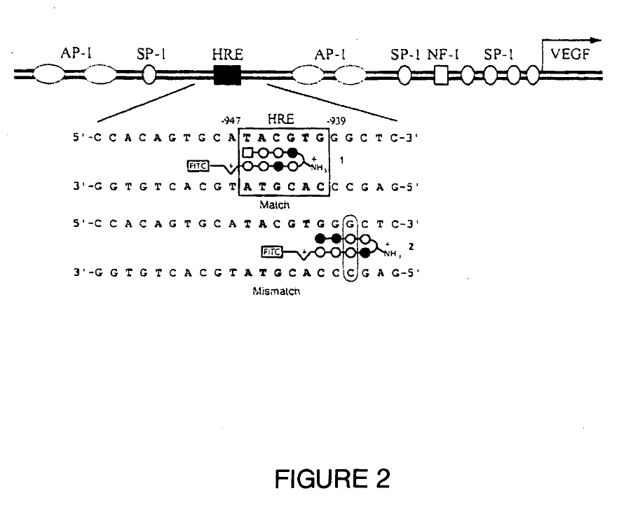

As described above, agents of the invention bind to an HRE of a hypoxia-inducible gene. Preferably the binding of the agent to the HRE reduces or inhibits the binding of an HIF to the HRE, thereby reducing or inhibiting the expression of a hypoxia-inducible gene in the cells under hypoxia conditions. Preferably, the agent binds to an HRE in the promoter of VEGF. In this respect, although the VEGF gene encodes multiple splicing variants, analysis of its promoter revealed that a single HRE is located between nucleotide positions -1000 to -900 (5′-TACGTG-3′) relative to the common transcription site (Tischer, et al., J. Biol. Chem., (1991), 266, 11947-11954). See FIG. 2. In certain preferred embodiments of the invention, the agent binds DNA that contains the sequence 5′-WTWCGW-3′, wherein W can be A or T. In preferred embodiments of the invention, the agent comprises a polyamide designed to bind DNA comprising the sequence 5′-WTWCGW-3′. In particularly preferred embodiments, the polyamide may include pyrrole and imidazole units. The DNA binding polyamide may optionally be conjugated to another molecule, for example a dye such as FITC. In certain embodiments, the DNA binding polyamide may be conjugated to a chlorothiophene. A particularly preferred example of such a pyrrole imidazole polyamide able to bind an HRE is polyamide 1, shown on in FIG. 1.

As used herein, the term “polyamide” refers to polymers of amino acids covalently linked by amide bonds (see, for example, U.S. Ser. No. 08/607,078, PCT/US97/03332, U.S. Ser. No. 08/837,524, U.S. Ser. No. 08/853,525, PCT/US97/12733, U.S. Ser. No. 08/853,522, PCT/US97/12722, PCT/US98/06997, PCT/US98/02444, PCT/US98/02684, PCT/US98/01006, PCT/US98/03829, and PCT/US98/0714 all of which are incorporated herein by reference in their entirety, including any figures). Preferably, the amino acids used to form these polymers include pyrrole, N-methylpyrrole, imidazole and N-methylimidazole. Polyamides containing pyrrole and imidazole amino acids are synthetic ligands that have an affinity and specificity for DNA comparable to naturally occurring DNA binding proteins. (See, e.g., Trauger, J. W., Baird, E. E. & Dervan, P. B. Nature, (1996), 382, 559-561; Swalley, S. E., Baird, E. E. & Dervan, P. B. J. Am. Chem. Soc., (1997), 119,6953-6961; Turner, J. M., Baird, E. E. & Dervan, P. B. J. Am. Chem. Soc., (1997), 119, 7636-7644; Trauger, J. W., Baird, E. E. & Dervan, P. B. Angewandte Chemie-International Edition, (1998), 37, 1421-1423; and Dervan, P. B. & Burli, R. W. Current Opinion in Chemical Biology, (1999), 3, 688-693).

The particular order of amino acids in such polyamides, and their pairing in dimeric, antiparallel complexes formed by association of two polyamide polymers, determines the sequence of nucleotides in dsDNA with which the polymers preferably associate. The development of pairing rules for minor groove binding polyamides derived from N-methylpyrrole and N-methylimidazole amino acids provided a useful code to control target nucleotide base pair sequence specificity. Specifically, an N-methylimidazole/N-methylpyrrole pair in adjacent polymers was found to distinguish G∘C from C∘G and both of these from A∘T or T∘A base pairs. A Py/Py pair was found to specify A∘T from G∘C but could not distinguish A∘T from T∘A. More recently, it has been discovered that inclusion of a new aromatic amino acid, 3-hydroxy-N-methylpyrrole (Hp) (made by replacing a single hydrogen atom in Py with a hydroxy group), in a polyamide and paired opposite Py enables A∘T to be discriminated from T∘A by an order of magnitude. Utilizing Hp together with Py and Im in polyamides provides a code to distinguish all four Watson-Crick base pairs (i.e., A∘T, T∘A, G∘C, and C∘G) in the minor groove of dsDNA as follows:

Pairing Code for Minor Groove Recognition

| Pair | G∘C | C∘G | T∘A | A∘T |
|---|---|---|---|---|
| Im/Py | + | − | − | − |
| Py/Im | − | + | − | − |
| Hp/Py | − | − | + | − |
| Py/Hp | − | − | − | + |

Favored (+),
disfavored (−)

As discussed above, a number of different polyamide motifs have been reported in the literature, including “hairpins,” “H-pins,” “overlapped,” “slipped,” and “extended” polyamide motifs. Specifically, hairpin polyamides are those wherein the carboxy terminus of one amino acid polymer is linked via a linker molecule, typically aminobutyric acid or a derivative thereof to the amino terminus of the second polymer portion of the polyamide. Indeed, the linker amino acid γ-aminobutyric acid (γ), when used to connect first and second polyamide polymer portions, or polyamide subunits, C→N in a “hairpin motif,” enables construction of polyamides that bind to predetermined target sites in dsDNA with more than 100-fold enhanced affinity relative to unlinked polyamide subunits. (See, for example, Turner, et al. J. Am. Chem. Soc., (1997), 119, 7636-7644; Trauger, et al. Angew. Chemie. Int. Ed. Eng., (1997), 37, 1421-1423; Turner, et al. J. Am. Chem. Soc., (1998), 120, 6219-6226; and Trauger et al. J. Am. Chem. Soc., (1998), 120, 3534-3535). Paired β-alanine residues (β/β), restore the curvature of the dimer for recognition of larger binding sites and in addition, code for AT/TA base pairs. (Trauger, J. W., Baird, E. E., Mrksich, M. & Dervan, P. B. J. Am. Chem. Soc., (1996), 118, 6160-6166; Swalley, S. E., Baird, E. E. & Dervan, P. B. Chem.-Eur. J., (1997), 3, 1600-1607; and Trauger, J. W., Baird, E. E. & Dervan, P. B. J. Am. Chem. Soc. (1998), 120, 3534-3535. Eight ring hairpin polyamides can bind a 6 base pair match sequence at subnanomolar concentrations with good sensitivity to mismatch sequences. Dervan, P. B. et al. Curr. Opin. Chem. Biol., (1999), 3: 688-693. Moreover, eight-ring hairpin polyamides (comprised of two four amino acid polymer portions linked C→N) have been found to regulate transcription and permeate a variety of cell types in culture (See Gottesfield, J. M. et al. Nature, (1997), 387:202-205.

An H-pin polyamide motif, i.e., wherein two paired, antiparallel polyamide subunits are linked by a linker covalently attached to an internal polyamide pair, have also been reported. Another polyamide motif that can be formed between linked or unlinked polyamide subunits is an “extended” motif, wherein one of the polyamide subunits comprises more amino acids than the other, and thus has a single-stranded region. See, for example, U.S. Ser. No. 08/607,078. In contrast, an “overlapped” polyamide is one wherein the antiparallel polyamide subunits completely overlap, whereas in a “slipped” binding motif, the two subunits overlap only partially, with the C-terminal portions not associating with the N-terminal regions of the other subunit. See U.S. Ser. No. 08/607,078.

Hairpin polyamide-dye conjugates enter the nucleus of cultured SW620 cancer cells and other cell lines in culture. (Best et al. Proc. Natl. Acad. Sci. USA, (2003), 100, 12063-68). Polyamide-chlorambucil conjugates blocked transcription by mammalian RNA polymerase II when the conjugates were targeted to the coding regions of genes, both in vitro and in cell culture, similar to the results reported for polyamide-duocarmycin conjugates. (Shinohara K et al. (2003), J. Am. Chem. Soc.; Oyoshi T et al., J Am Chem Soc (2003), 125, 4752-4). Polyamides, such as pyrrole imidazole polyamides, which are useful alone, or as conjugates, can be prepared as described (see U.S. Pat. No. 6,559,125, which is incorporated herein by reference).

The pyrrole imidazole polyamides are a class of small molecules that can be designed to bind predetermined DNA sequences (See, e.g., Dervan, P. B., Bioorgan. Med. Chem. (2001) 9, 2215-2235; Dervan, et al., Curr. Opin. Struct. Biol. (2003), 13, 284-299; Marques et al., J. Am. Chem. Soc. (2004), 126, 10339-10349; Renneberg et al., J. Am. Chem. Soc., (2003), 125, 5707-5716; Foister et al., Bioorg. Med. Chem. II, (2003), 4333-4340; Doss et al., Chemistry & Biodiversity (2004), 1, 886-899; Briehn et al., Chem. Eur. J. (2003), 9, 2110-2122; U.S. Pat. No. 6,559,125, and U.S. application Ser. No. 11/038,506, filed Jan. 18, 2005). These molecules bind their target sites in genomic DNA with affinities comparable to natural DNA-binding transcription factors. (Dudouet, B. et al. Chem Biol., (2003), 10, 859-67). Additionally, polyamides can optionally be conjugated with an alkylator which allows the polyamide to deliver an alkylation warhead to pre-determined sites in genomic DNA in the cell nucleus. (Wurtz, et al. Chem. & Biol. (2000), 7, 153-161). Inhibition of transcription in vitro (Oyoshi, et al. J Am Chem Soc (2003), 125, 4752-4) and luciferase expression in mammalian cell culture transfection experiments has been obtained with polyamide-alkylator (duocarmycin DU86) conjugates. (Shinohara, K. et al. (2004) J. Am. Chem. Soc.). As demonstrated herein, polyamides without alkylator conjugates can also be effective in inhibiting the expression of certain genes.

Accordingly, a method for interfering with a hypoxia mediated transcriptional pathway of a cell is provided by the present invention. Generally, the method involves contacting a cell with any of the agents as described above. Preferably, the agent interferes with the function of any one or more of the components of a hypoxia mediated transcriptional pathway. The components of the hypoxia mediated transcriptional pathway can be selected from an HRE, an HIF, a hypoxia-inducible gene and any relevant co-transcriptional factors, binding partners, or combinations thereof. Interference with the hypoxia mediated transcriptional pathway may cause a reduction or inhibition in expression of a hypoxia-inducible gene. In another embodiment, the agent binds specifically to an HRE. Preferably the binding of the agent to the HRE reduces or inhibits the expression of a hypoxia-inducible gene.

The present invention also provides methods for disrupting the binding between an HRE and an HIF. Generally, the methods involve contacting an the cells of an HRE with any of the agents as described above. Contacting an HRE with an agent causes binding between the agent and HRE, thereby disrupting binding between the HRE and HIF. In another embodiment, the disruption of the binding between the HRE and HIF may lead to reduced expression of a hypoxia inducible gene.

The present invention also provides methods for reducing or inhibiting expression of a hypoxia-inducible gene in a cell under a hypoxia condition. Generally, the methods involve contacting the hypoxia-inducible gene with any of the agents as described above. The agents bind to the HRE of the promoter region of the gene, thereby reducing or inhibiting interaction of the HRE with the HIF-1α/ARNT heterodimer. The binding of the HIF-1α/ARNT heterodimer with the HRE (O'Rourke, et al., Eur. J. Biochem., (1996), 241 403-410) creates favorable conditions for the recruitment of p300/CBP and SRC-1 family co-activators which drive the expression of hypoxia-inducible genes. In certain embodiments, contacting the HRE of the hypoxia-inducible gene with the agent of the invention reduces or inhibits expression of one or more hypoxia-inducible genes. In another embodiment, the reduction of the HIF-1α/ARNT heterodimer activity may result from the reduction of mRNA levels of the genes that code for the hypoxia response element. Such a reduction of mRNA levels could, for example, occur as a result of inhibition or impairment of the transcription of the gene encoding the mRNA of interest; or by posttranscriptional effects such as the degradation of mRNA transcripts or the impairment of translation.

The present invention also provides methods for reducing or inhibiting growth of tumors, more specifically, methods for reducing or inhibiting the growth of solid tumors. Generally the methods involves contacting a tumor with any of the agents as described above. The contacting of the tumor with the agents of the invention results in an interference with the hypoxia-mediated transcriptional pathway of the tumor. In one embodiment, the method for reducing or inhibiting tumor growth involves contacting the HRE of a hypoxia-inducible gene associated with a tumor with the agent of the invention. The agent binds to the DNA sequence encoding the HRE, thereby preventing the HIF-1α/ARNT heterodimer from binding with the HRE and reducing or inhibiting expression of the hypoxia-inducible gene. In some embodiments of the method, the agent binds the HRE of a VEGF gene.

Preferably, the methods for reducing and inhibiting growth in tumors described above with agents of the present invention causes tumor growth rate to be decreased by at least about 25%, or at least 50%, or at least 75%. More preferably, tumor growth rate is completely inhibited. In certain preferred methods of treating tumors, the tumor size, as measured by methods known in the art in terms of either mass or volume, is reduced relative to the size of the tumor prior to contacting with any agent of the present invention. A reduction of tumor size can be any detectable reduction in the size of the tumor as compared to the tumor prior to contacting with the agent; however, preferably the agent causes the tumor to be decreased in size by at least about 25%, or at least 50%, or at least 75%. More preferably, the tumor is reduced in size below detectable limits.

The present invention also provides methods for reducing or inhibiting metastatic spread of tumors in a patient. Generally, the method involves administering any agent of the invention, as provided above, to a patient with a tumor. The tumor cells may be in a hypoxia condition. In some embodiments, the tumor may be a solid tumor. Methods for determining the effectiveness reducing metastatic spread are known to the skilled clinician. For example, imaging methods can be used to determine whether a patient's tumor has increased or decreased in size. Preferably, an agent of the present invention binds an HRE of a hypoxia inducible gene. In a preferred embodiment, the hypoxia inducible gene is VEGF. The binding of the agent to the HRE may disrupt a hypoxia mediated transcriptional pathway, such as for example, the binding of HRE and HIF. In preferred embodiments of the method, the disruption may cause a reduction or inhibition of expression in hypoxia-inducible genes.

In related embodiments, the invention involves a method for reducing or inhibiting angiogenesis within a tumor under hypoxia conditions, wherein the tumor has elevated levels of expression of a hypoxia inducible gene. As such, in one embodiment the method may involve contacting the tumor cell of the tumor with an agent that reduces mRNA of genes encoding angiogenic peptides or glucose metabolism protein levels, wherein, prior to the contacting step, the tumor cell has expression levels of a hypoxia inducible gene that are greater than the level in corresponding normal cells. In certain preferred embodiments, the hypoxia inducible gene is VEGF.

In certain embodiments, the method may include contacting a tumor cell with an agent that binds to a hypoxia response element. As used herein the term “hypoxia response element gene” includes any gene that contains a hypoxia response element. As such, the agent of the method may bind to DNA of a hypoxia response element gene. The term “binding” as used herein in the context of an agent binding to DNA broadly refers to any chemical interaction between the agent and the particular DNA of interest. One example of binding includes interactions between polyamide molecules and a target DNA (see below and see also U.S. Pat. No. 6,559,125). In a preferred embodiment, the polyamide may be a pyrrole imidazole polyamide, such as for example, polyamide 1 shown in FIG. 1. Preferably, the binding of an agent to DNA of a hypoxia response element in a cell, either directly or indirectly, results in a reduction or inhibition of VEGF activity in the cell, which in turn results in a reduction or inhibition of proliferation and angiogenesis of the tumor cell.

In preferred embodiments, agents of the invention bind DNA of a hypoxia response element in the promoter of a hypoxia inducible gene. For example, as shown in FIG. 2, in certain embodiments, the agent of the invention binds to an HRE in the promoter of the VEGF gene having the sequence 5′-WTWCGW-3′, where W can be A or T. In certain preferred embodiments the agent can bind the DNA of the hypoxia response element of the VEGF gene between nucleotide positions 1330 to 1430 of the VEGF gene represented by Accession Number AF095785, preferably between nucleotide positions 1350 to 1420, more preferably between nucleotide positions 1375 and 1400, and even more preferably between nucleotide positions 1388 and 1393.

In certain preferred embodiments, the agent binds to the DNA of interest in the target cell and causes a chemical modification of the DNA. Examples of such chemical modifications may include, but are not limited to, alkylation or degradation of the DNA. As such, in certain embodiments, an agent of the invention may be conjugated to a chemotherapeutic molecule, such as for example, an alkylator.

DNA alkylators were among the first anti-cancer drugs developed and are the most commonly used agents in cancer chemotherapy. (Zewail-Foote, et al., Anticancer Drug Des (1999), 14, 1-9). Alkylators induce cross-linking of DNA strands, abnormal base pairing, or DNA strand breaks, thus blocking cells in the G2/M phase of the cell cycle, thereby preventing cancer cell proliferation. Since conventional alkylators modify DNA at numerous sites in the genome, considerable effort has been expended to devise more sequence-specific alkylators, in the hope that increasing DNA sequence specificity will decrease the unwanted side effects of nonspecific alkylators, while retaining the ability of the compound to kill cancer cells. Two approaches that have been taken are development of DNA alkylators with some degree of DNA sequence specificity, such as the duocarmycins and pyrrolobenzodiazepines (Boger, et al., Bioorg Med Chem Lett, (2000), 10, 495-8; Gregson, et al., J Med Chem (2001),44, 737-48), and linking existing alkylators, such as chlorambucil or the duocarmycins with more sequence-specific DNA-binding small molecules. (Wurtz, et al., Chem. & Biol. (2000), 7, 153-161; Shinohara, et al., J. Am. Chem. Soc. (2004)). A number of small molecule-alkylator conjugates have been explored as potential cancer therapeutics. One agent that has been used in cancer chemotherapy is the bifunctional alkylator tallimustine, a synthetic derivative of tri-pyrrole distamycin A, in which the NH₂-terminal formyl group is substituted by the benzoyl mustard chlorambucil (Chl). Studies in SCID mice using human leukemic cell lines demonstrated that tallimustine is effective in prolonging survival, and in producing some cures in mice. Following these observations, a phase I-II study of tallimustine in acute leukemia and other advanced cancers was initiated. (Weiss, G. R. et al. Clin Cancer Res (1998), 4, 53-9. However, tallimustine is dose-limited by myelosuppression because of a lack of specificity.

A small organic molecule useful for inhibiting hypoxia inducible gene expression in cells according to a method of the invention is exemplified by a polyamide, such as for example, pyrrole imidazole polyamide 1. In one aspect, the pyrrole imidazole polyamide comprises a conjugate, which can include a chemotherapeutic molecule operatively linked to the pyrrole imidazole polyamide. Such a conjugate is exemplified, for example, by a pyrrole imidazole polyamide having a DNA alkylator (such as, for example, chlorambucil) operatively linked thereto. Examples of conjugated pyrrole imidazole polyamides as well as methods for designing conjugated pyrrole imidazole polyamides that could be useful in the present invention are disclosed in U.S. Pat. No. 6,559,125, which is incorporated herein by reference.

The present invention also provides methods for treating a patient with a tumor by administering an agent that reduces or inhibits expression of a hypoxia inducible gene. Efficacy is identified by detecting for signs or symptoms associated with a decrease in the expression of the gene under hypoxia conditions. The signs and symptoms characteristic of particular types of tumors are well known to the skilled clinician, as are methods for monitoring the signs and conditions. For example, imaging methods can be used to determine whether a tumor has increased or decreased in size, or is increasing in size at a reduced growth rate, due to treatment according to the present methods.

The invention also provides a method of determining whether a tumor is susceptible to treatment with an agent that reduces or inhibits hypoxia inducible gene expression. Such a method can be performed by determining that the level of hypoxia inducible gene expression in a tumor cell sample for the individual is decreased compared to the level of gene expression in corresponding normal cells. In certain preferred embodiments the hypoxia inducible gene is VEGF. The level of gene expression can be determined using methods as disclosed herein or otherwise known in the art. Likewise, the invention provides a method for determining whether a tumor is susceptible to treatment with an agent that reduces or inhibits activity of a hypoxia inducible gene other than VEGF. The method is performed by evaluating the expression of hypoxia inducible genes in a tumor cell, and when gene expression is decreased as compared with gene expression in corresponding normal cells, the tumor is likely to be susceptible to treatment with an agent that reduces or inhibits the expression of the hypoxia inducible gene.

An agent of the invention may be formulated as a pharmaceutically acceptable salt or complex or with a pharmaceutically acceptable carrier. The form of the composition will depend, in part, on the route by which the composition is to be administered. Generally, the composition will be formulated such that the agent is in a solution or a suspension, such a form be suitable for administration by injection, infusion, or the like, or for aerosolization for administration by inhalation. However, the composition also can be formulated as a cream, foam, jelly, lotion, ointment, gel, or the like, or in an orally available form.

A pharmaceutically acceptable carrier useful for formulating an agent for use in a method of the invention can be aqueous or non-aqueous, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the agent, for example, carbohydrates, such as glucose, sucrose or dextrans, antioxidants, such as ascorbic acid or glutathione, chelating agents, low molecular weight proteins or other stabilizers or excipients.

The pharmaceutical composition also can comprise an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary stabilizing, thickening or coloring agents can be used, for example a stabilizing dry agent such as triulose.

The amount of the particular agent contained in a composition can be varied, depending on the type of composition, such that the amount present is sufficient reduce or inhibit hypoxia induced gene expression, and more specifically, an amount sufficient to reduce or inhibit VEGF expression, as appropriate, thereby treating the tumor patient. In general, an amount of an agent sufficient to provide a therapeutic benefit can be determined using routine clinical methods, including Phase I, II and III clinical trials.

The method may be performed in a high throughput format, thus facilitating the examination of a plurality of tumor cell samples, which can be the same or different or a combination thereof, in parallel. As such, the method allows for detecting the level of hypoxia response element gene expression in a plurality of samples, including 1, 2, 3, 4, 5, or more tumor samples and, as desired, 1, 2, 3, 4, 5 or more control samples. In another embodiment, the method is performed in a multiplex format, wherein the level of hypoxia response element gene expression is detected in at least a second tumor sample, or in at least a first corresponding normal cell sample, or in a combination thereof. Methods of performing multiplex assays in a high throughput format also are provided.

For a high throughput format, samples, which can be samples of tumor cells, of extracts of the tumor cells, or of nucleic acid molecules (e.g., RNA) isolated from the tumor cells, the samples can be deposited manually or robotically on a solid support (e.g., a glass slide or a silicon chip or wafer). Generally, the samples are arranged in an array or other reproducible pattern, such that each sample can be assigned an address (i.e., a position on the array), thus facilitating identification of the source of the sample. An additional advantage of arranging the samples in an array, particularly an addressable array, is that an automated system can be used for adding or removing reagents from one or more of the samples at various times, or for adding different reagents to particular samples. In addition to the convenience of examining multiple samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

The following examples are intended to illustrate but not limit the invention.

### EXAMPLES

### Example 1

### Synthesis of Polyamides

Polyamides 1 and 2 (FIG. 1) were synthesized by solid-phase methods of Kaiser oxime resin (Nova Biochem) (Belitsky, et al. (2002) Bioorg. Med. Chem. 10:2767-2774) and conjugated to FITC isomer I (Best, et al. (2003) Proc. Natl. Acad. Sci. USA 100:12063-12068). The purity and identity of the polyamide-dye conjugates were verified by analytical HPLC, UV-visible spectroscopy, and MALDI-ToF MS.

### Example 2

### Determination of DNA-Binding Affinities and Sequence Specificities of Polyamides 1 and 2

A 5′ ³²P-labeled fragment was generated by PCR amplification of the site from the plasmid pGL2-VEGF-Luc by using primers 5′-CTC AGT TCC CTG GCA ACA TCT-3′ (VEGFP1) and 5′-TGG CAC CAA GTT TGT GGA GCT-3′ (VEGFP2) and isolated by nondenaturing gel electrophoresis (Trauger, et al. (2001) Methods Enzymol. 340:450-466). Quantitative DNase I footprint titration experiments were used to determine the binding affinities and specificities of polyamides 1 and 2 (Trauger, et al.).

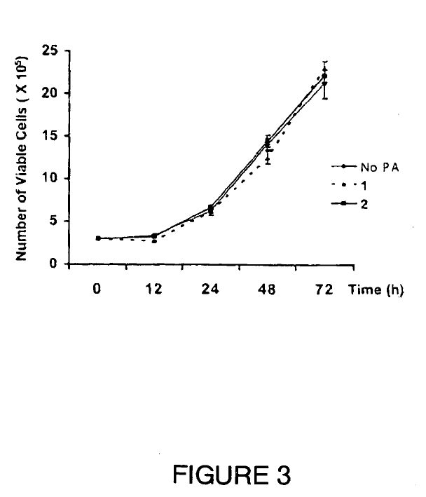

Based on the pairing rules, match polyamide 1 targets sequences of the type 5′-WTWCGW-3′ (where W=A or T), whereas mismatch polyamide 2 targets sequences of the type 5′-WGGWCW-3′. The 3-chlorothiophene ring at the N terminus of polyamide 1 provides specificity for a T·A base pair (Foister, et al. (2003) Bioorg. Med. Chem. 11:4333-4340). The detailed binding sites were mapped for both match and mismatch polyamides on the VEGF promoter fragment that encompasses the HRE. From DNase I footprint titrations, a Kₐ value of 6.3×10⁹ M⁻¹ was obtained for polyamide 1 at the HRE site (FIG. 3A). The mismatch polyamide 2 bound the HRE site with ≈100-fold lower affinity (Kₐ=7.9×10⁷M⁻¹). No match sites at 1.0 nM concentration could be found for polyamide 2 in the region of this DNA that can be resolved by gel electrophoresis.

### Example 3

### Disruption of the HIF-DNA Complex

The HIF1α/ARNT heterodimer was transcribed/translated in vitro by using Promega TNT kit according to the manufacturer's instructions. The double-strand oligonucleotide probe was prepared by annealing the two complementary strands 5′-GAC TCC ACA GTG CAT ACG TGG GCT CCA ACA GGT-3′ (HRE-EMSA1) and 5′-ACG TGT TGG AGC CCA CGT ATG CAC TGT GGA GTC-3′ (HRE-EMSA2). Before annealing, the HRE-EMSA1 oligonucleotide was 5′-end radiolabeled with γ-³²-^{P}-ATP (NEN) and T4 polynucleotide kinase, as described. The radiolabeled double-strand oligonucleotide probe was isolated by using a G25 Quickspin column (Boehringer Mannheim).

Polyamides were pre-incubated with the radiolabeled oligonucleotide in Z-buffer (100 mM KCl/25 mM Tris, pH 7.5/0.2 mM EDTA/20% glycerol/0.25 mg/ml BSA/0.05% Nonidel P-40/5 mM DTT/0.1 mg/ml PMSF/1.2 mM sodium vanadate) at 0° C. for 30 minutes. Then, the in vitro transcribed/translated protein mixture, diluted with the same buffer, was added and the mixture was held on ice for an additional 30 min. Each time, the following controls were included: free oligonucleotide probe, probe with unprogrammed in vitro transcription/translation reaction mixture, and 100-fold excess of competing non-radiolabeled probe. The complexes were resolved on a 4% non-denaturing polyacrylamide gel and visualized with the Strom 820 Phosphorimager (Molecular Dynamics).

### Example 4

### Uptake of Polyamides in Cultured Cells

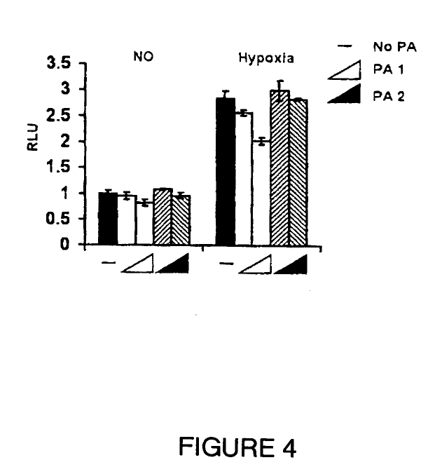

The uptake of both polyamides by the HeLa cell line was examined by laser-scanning confocal microscopy. Previous studies indicated that the degree of cellular uptake and nuclear localization of polyamides containing an eight-ring sequence recognition core depends on the pyrrole/imidazole content of the core and varies for each cell line (Best, et al. (2003) Proc. Natl. Acad. Sci. USA 100: 12063-12068; Edelson, et al. (2004) Nucleic Acids. Res. 32:2802-2818). It was found that both polyamides exhibit strong nuclear localization after incubation at 2 μM concentration for 12 h at 37° C. in standard culture medium (FIG. 4).

HeLa cells were trypsinized for 5-10 min at 37° C., centrifuged for 5 min at 5° C. at 2,000 rpm in a Beckman-Coulter Allegra 6R centrifuge, and re-suspended in a fresh medium to a concentration of 1.25×10⁶ cells per ml. Incubations were performed by adding 150 μl of cells into culture dishes equipped with glass bottoms for direct imaging (MatTek, Loveland, Ohio). The cells were grown in the glass-bottom culture dishes for 24 h. The medium was then removed and replaced with 142.5 μl of fresh medium. Then 7.5 μl of the 100 μM polyamide solution was added and the cells were incubated in a 5% CO₂ atmosphere at 37° C. for 10-14 h. Imaging was performed on a Zeiss LSM 5 Pascal inverted laser scanning microscope equipped with a ×40 oil-immersion objective lens. Analysis of the images was performed as previously described by Best, et al. (2003) Proc. Natl. Acad. Sci. U.S.A. 100:12063-12068.

### Example 5

### Effect of Polyamides on Cell Viability and Growth Rate

HeLa cells were incubated with polyamides at 1 μM concentration, trypsinized and counted at various time points (0-72 h) by using a hemacytometer to determine the effects of prolonged incubation on cell viability. Measurements of cell growth rates indicate that polyamides at 1 μM in standard culture medium have no deleterious effects on cell growth and division, as shown in FIG. 3. The data represents results from three independent experiments with error bars indicating mean standard deviation. Briefly, 1.5×10⁵ HeLa cells were split into six-well tissue culture plates 24 h prior to incubation with polyamides. The cells were trypsinized after 0, 12, 24, 48, and 72 counted using a hemocytometer.

### Example 6

### Analysis of Promoter Activity with Luciferase Assays

In the luciferase experiments, HeLa cells that had been stably transfected with a reporter plasmid VEGF-Luc containing the VEGF promoter upstream of luciferase CDNA. The experiments were carried out in a hypoxic chamber with 1% O₂ to mimic closely the conditions of physiological hypoxia. Incubation with the match polyamide 1 resulted in a decrease of promoter activity in a dose-dependent manner, as indicated by decreased levels of luciferase activity. As shown in FIG. 4, a negligibly small effect was observed for mismatch polyamide 2.

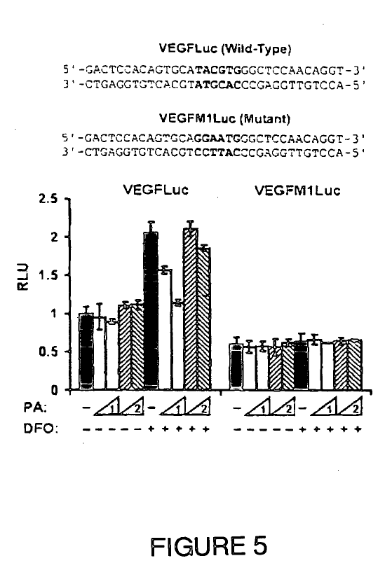

As a specificity control, VEGF-M1Luc, a nearly identical reporter in which the HRE and surrounding sequences were mutated to disfavor HIF 1 binding, was constructed and used in parallel. FIG. 5 shows the inhibition of the expression of wild-type and mutated VEGF-Luc under hypoxia conditions. Hypoxia mimetic compound DFO (Woods, et al., Int. J. Biochem. Cell Biol., (1997), 29, 1419-1432; Bianchi, et al., Nucleic Acids. Res., (1999), 27, 4223-4227) was used to stabilize HIF and activate HIF target genes. Cells were harvested after incubation with 300 μM DFO for 12-16 h.

Treatment of the stably transfected HeLa cells with polyamide 1 resulted in significant attenuation of the hypoxia inducible VEGF-Luc activity. By contrast, treatment of the transfected HeLa cells with polyamide 2 resulted in only a modest decrease in VEGF-Luc activity. The VEGF-M1Luc promoter with a mutated HRE site showed no inducibility under hypoxic conditions and both polyamide 1 and 2 had no effect on the levels of luciferase activity in the mutant promoter, as shown in FIG. 5. No obvious cytotoxicity was observed in any of the experiments.

### Example 7

### Suppression of Hypoxia-Inducible Transcription in Cultured Cells

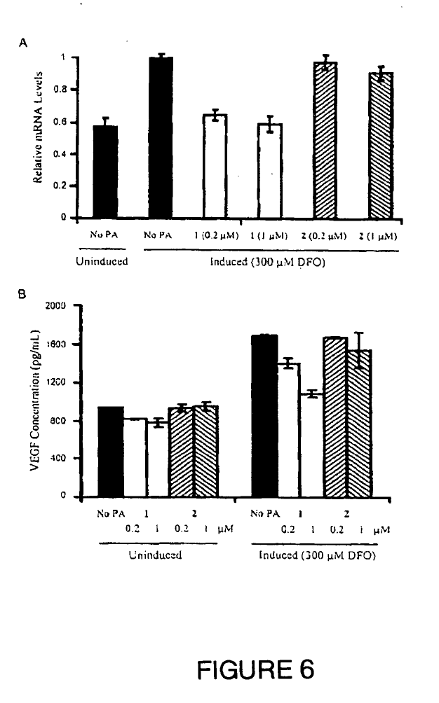

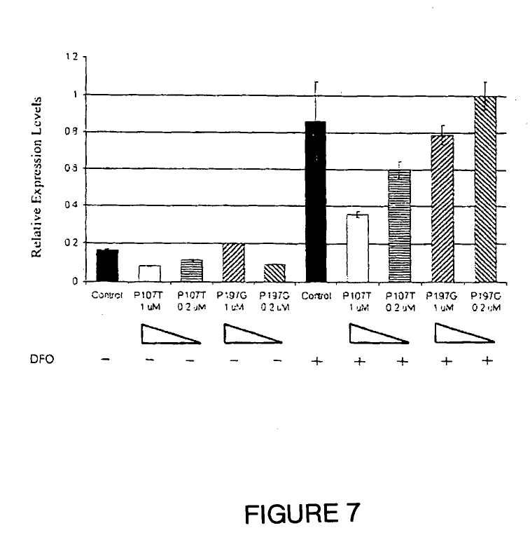

Real-time quantitative RT-PCR analysis was used to evaluate the relative levels of VEGF in mRNA in hypoxic HeLa cells treated with the polyamides. In parallel, untreated cells were used as controls. Expression of β-glucuronidase was used as a control gene for determining the relative levels of transcription (Hung, et al., J. Clin. Endocrinol. Metab., (2003), 88, 3694-3699). After 48 h of incubation with polyamide 1, levels of VEGF expression were reduced in a dose dependent manner, see FIG. 6a. Polyamide 1 at 1 μM inhibits approximately 60% of VEGF expression, which is near the VEGF mRNA levels in the uninduced (normoxic) cells. Mismatch polyamide 2 shows minimal inhibition at either 0.2 μM or 1 μM concentrations. FIG. 7 shows dose dependent reduction of expression, as measures from real-time PCR, under normoxia and hypoxia conditions.

ELISA was used to determine the levels of secreted VEGF. Total protein levels were monitored in parallel, to exclude the possibility of disruption of general transcriptional activity by the polyamides. Under normoxia, match polyamide 1 caused a modest decrease of the basal expression levels of VEGF, whereas mismatch polyamide 2 caused no decrease of VEGF levels. Under hypoxia conditions, polyamide 1 decreased levels of VEGF in a dose-dependent manner, whereas mismatch polyamide 2 had a minimal effect. See FIG. 6B.

### Example 8

### Genome Wide Effects of Polyamides

The effects of polyamide treatment on nuclear transcription were monitored by global gene expression analysis using Affymetrix high-density UniGene 133A arrays, which contain oligonucleotides representing over 20,000 human genes. HeLa cells were treated in triplicate with no polyamide, polyamide 1, or polyamide 2 at 1 μM and 0.2 μM concentrations for 48 h, which is a sufficient time for transcription. Hypoxia conditions were induced by adding DFO and exposing to further incubation.

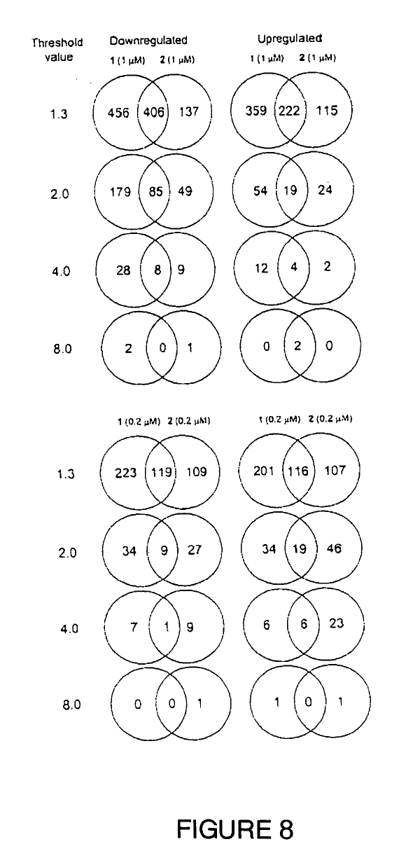

The number of genes uniquely and similarly affected by polyamides 1 and 2 at concentrations of 0.2 μM and 1.0 μM are shown in FIG. 8. Significant overlap of affected genes for polyamides 1 and 2 exists at lower threshold values, and decreases with increasing threshold values. These results are consistent with previous work suggesting that polyamides targeting different DAN sequences can affect expression of different sets of genes (Dudouet et al., Chem. Biol., 10 (2003), 859-867). At a threshold of 2.0-fold, 264 and 73 genes are downregulated and upregulated, respectively, in the presence of polyamide 1 at 1 μM. This represents only 1.5% of the interrogated genes. In the case of polyamide 2, less than 1% are affected at this threshold. These effects are surprising, given that a 6-bp binding site is expected to have greater than 1.4 million match sites in a 3 billion-base pair genome. Genes affected at a threshold of 2.0-fold for each polyamide are listed in Tables 2 and 3, which are published as supporting information on the PNAS site for Olenyuk, et al., PNAS, (2004) 101, 16768-16773, and is incorporated herein by reference. Polyamides 1 and 2 at 0.2 μM affect the expression of fewer genes are each threshold level as compared with the 1 μM data sets. In should be noted that most genes down- and up-regulated by each polyamide at 0.2 μM are similarly affected in the 1 μM data set for each polyamide.

Differential expression levels of several hypoxia-inducible genes in the presence of polyamide 1 or 2 are provided in Table 1. Expression of the VEGF is down regulated by 1.34-fold with 1.0 μM polyamide 1 and 1.4-fold with 0.2 μM polyamide 2. VEGF expression is unchanged with 0.2 μM and 1.0 μM polyamide 2. This data correlates well with the RT-PCR and luciferase experiments. Other hypoxia inducible genes are also affected, albeit to a different extent. Microarray data indicates significantly down regulated levels of the mRNAs corresponding to all endothelin genes, and particularly a 13.6-fold (greater than 90%) down regulation of gene expression in Endothelin-2 as compared with the untreated controls. Polyamide 2 at a concentration of 1.0 μM demonstrated a 2.8-fold down regulation of Endothelin-2. This was validated by real-time quantitative RT-PCR of endothelin-2 mRNA levels, where 6.8-fold down regulation was observed for polyamide 1 and 2.4-fold for polyamide 2. According to the microarray data, endothelin-1 was found to be down regulated 2.4-fold by polyamide 1 and 1.26-fold by polyamide 2. Real-time quantitative RT-PCR measurements were generally consistent with a 1.5-fold down regulation of endothelin-1 mRNA by polyamide 1 and no detectable down regulation by polyamide 2. Recent studies have indicated endothelins may play an important role in cancer (Nelson, et al., Nat. Rev. Cancer, (2003), 3, 110-116). In addition, endothelin-2 has been recently implicated as an autocrine survival factor in cells in hypoxia conditions (Grimshaw, et al., Mol. Cancer Ther., (2002), 1, 1273-1281).

TABLE 1
Relative expression levels of selected hypoxia inducible
genes in the presence of polyamides 1 and 2.

| | | Fold change | Fold change | Fold change | Fold change |
|---|---|---|---|---|---|
| Annotated | GenBank | with 1.0 μM | with 0.2 μM | with 1.0 μM | with 0.2 μM |
| Gene | Accession No. | of polyamide 1 | of polyamide 1 | of polyamide 2 | of polyamide 2 |
| Hexokinase-2 | AI761561 | −1.3 | −1.1 | Unchanged* | Unchanged* |
| Aldolase-C | NM_005165.1 | −1.1 | −1.2 | Unchanged* | Unchanged* |
| Erythropoietin (EPO) | | +1.1 | +1.7* | +2.1 | Unchanged* |
| Transforming | J03241.1 | +1.1 | +1.1* | +1.1 | Not determined |
| Growth Factor β3 | | | | | |
| Endothelin-1 | NM_001955.1 | −2.4 | −1.9* | −1.3 | −2.0* |
| Endothelin-2 | NM_001956.1 | −13.2 | −2.2 | −2.8 | −2.0 |
| Endothelin-3 | NM_000114.1 | −1.8 | −1.2 | Unchanged | −1.3 |
| VEGF | AF022375.1 | −1.35 | −1.4 | Unchanged | Unchanged* |
| VEGF Receptor, Flt 1 | NM_002019.1 | −1.5 | −1.6* | −1.1* | −1.4* |

Although the invention has been described with reference to the above example, it will be understood that modifications and variations are encompassed within the spirit and scope of the invention. Accordingly, the invention is limited only by the following claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A composition comprising a DNA binding polyamide or a pharmaceutically acceptable salt or complex thereof, wherein said composition or pharmaceutically acceptable salt or complex thereof is able to bind to a hypoxia response element in a cell.

2. The composition of claim 1 wherein said DNA binding polyamide or a pharmaceutically acceptable salt or complex thereof is able to bind to a hypoxia response element in a cell and reduce expression of a hypoxia inducible gene associated with said response element.

3. The composition of claim 1 wherein said DNA binding polyamide or a pharmaceutically acceptable salt or complex thereof is able to reduce or inhibit the binding of hypoxia-inducible factor-1a/aryl hydrocarbon receptor nuclear translocator (HIF-1α/ARNT) to said hypoxia response element when said DNA binding polyamide or a pharmaceutically acceptable salt or complex thereof is bound to said hypoxia response element.

4. The composition of claim 1 wherein said DNA binding polyamide or a pharmaceutically acceptable salt or complex thereof is able to reduce the expression of genes encoding angiogenic peptides when said DNA binding polyamide or a pharmaceutically acceptable salt or complex thereof is bound to said hypoxia response element associated with genes encoding angiogenic peptides in a cell.

5. The composition of claim 1, wherein the polyamide binds DNA comprising the sequence 5′-WTWCGW-3′, wherein W is A or T.

6. The composition of claim 1, wherein said DNA binding polyamide comprises N-methylpyrrole and N-methylimidazole subunits.

7. The composition of claim 1, wherein said DNA binding polyamide is polyamide 1.

8. A method for interfering with a hypoxia-mediated transcriptional pathway in a cell comprising contacting said cell with an agent that binds to a hypoxia response element.

9. The method of claim 8, wherein said interference reduces or inhibits binding of a hypoxia inducible factor to said hypoxia response element.

10. The method of claim 8, wherein said interference reduces expression of a hypoxia inducible gene.

11. The method of claim 10, wherein said hypoxia-induced gene is selected from the group consisting of vascular endothelial growth factor (VEGF), VEGF receptor-1, platelet-derived growth factor B, hexokinase-1 and -2, aldolase-A and -C, erythropoietin, endothelin 1, endothelin 2, endothelin 3, nitric oxide synthase-2, heme oxygenase-1, ceruloplasmin, transferrin, transferrin receptor, insulin-like growth factor-binding protein-1, -2 and -3, insulin-like growth factor II, transforming growth factor-β3, cyclooxygenase-1, phosphoglycerate kinase-1, phosphofructokinase, glucose transporters 1 and 3, glyceraldehydes-3-phosphate dehydrogenase, enolase 1, pyruvate kinase M, lactate dehydrogenase A, and adenylate kinase 3.

12. The method of claim 8, wherein said agent binds to a hypoxia response element in said cell thereby reducing expression of an associated hypoxia induced gene, wherein said reduced expression in said cell occurs more effectively under hypoxia conditions than under normoxia conditions.

13. The method of claim 8, wherein said cell is under hypoxia conditions.

14. The method of claim 8, wherein said agent is a pyrrole imidazole polyamide.

15. The method of claim 8, wherein said agent is polyamide 1.

16. The method of claim 8, wherein said agent binds DNA comprising the sequence of 5′-WTWCGW-3′, wherein W is A or T.

17. A method for reducing the growth and/or metastatic spread of a tumor comprising contacting said tumor with an agent that binds to a DNA sequence encoding a hypoxia response element.

18. The method of claim 17 wherein said agent reduces or inhibits expression by a hypoxia inducible gene.

19. The method of claim 18, wherein said hypoxia inducible gene is selected from the group consisting of vascular endothelial growth factor (VEGF), VEGF receptor-1, platelet-derived growth factor B, hexokinase-1 and -2, aldolase-A and -C, erythropoietin, endothelin 1, endothelin 2, endothelin 3, nitric oxide synthase-2, heme oxygenase-1, ceruloplasmin, transferrin, transferrin receptor, insulin-like growth factor-binding protein-1, -2 and -3, insulin-like growth factor II, transforming growth factor-β3, cyclooxygenase-1, phosphoglycerate kinase-1, phosphofructokinase, glucose transporters 1 and 3, glyceraldehydes-3-phosphate dehydrogenase, enolase 1, pyruvate kinase M, lactate dehydrogenase A, and adenylate kinase 3.

20. The method of claim 18, wherein said hypoxia inducible gene is VEGF.

21. The method of claim 20, wherein said agent binds the DNA of the hypoxia response element between nucleotide positions -1000 to -950 of the VEGF gene relative to the common transcription site.

22. The method of claim 20, wherein said agent binds the DNA of the hypoxia response element between nucleotide positions -975 and -970 of the VEGF gene relative to the common transcription site.

23. The method of claim 17, wherein said DNA comprises the sequence 5′-WTWCGW-3′, wherein W is A or T.

24. The method of claim 17, wherein said agent comprises a pyrrole imidazole polyamide.

25. The method of claim 17, wherein said agent is polyamide 1.


SEQUENCE LISTING (6 sequences)

SEQ ID NO: 1 - DNA, 21 bp, Artificial Sequence
  Description of Artificial Sequence Synthetic primer
ctcagttccctggcaacatct

SEQ ID NO: 2 - DNA, 21 bp, Artificial Sequence
  Description of Artificial Sequence Synthetic primer
tggcaccaagtttgtggagct

SEQ ID NO: 3 - DNA, 33 bp, Homo sapiens
gactccacagtgcatacgtgggctccaacaggt

SEQ ID NO: 4 - DNA, 33 bp, Homo sapiens
acgtgttggagcccacgtatgcactgtggagtc

SEQ ID NO: 5 - DNA, 21 bp, Homo sapiens
ccacagtgcatacgtgggctc

SEQ ID NO: 6 - DNA, 33 bp, Homo sapiens
gactccacagtgcaggaatgggctccaacaggt